(19) United States
(12) Patent Application Publication
(10) Pub. No.: US 2016/0074308 A1
(43) Pub. Date: Mar. 17, 2016

(54) METHOD FOR PRODUCING A COSMETIC CLEANING AGENT

(71) Applicant: Henkel AG & Co. KGaA, Duesseldorf (DE)

(72) Inventors: Jutta Franklin, Solingen (DE); Barbara Heide, Krefeld (DE); Thomas Foerster, Duesseldorf (DE)

(21) Appl. No.: 14/951,775

(22) Filed: Nov. 25, 2015

Related U.S. Application Data
(63) Continuation of application No. PCT/DE2014/200222, filed on May 19, 2014.

(30) Foreign Application Priority Data
May 28, 2013 (DE) 10 2013 209 895.2

(51) Int. Cl. A61K 8/85; A61K 8/362; A61Q 19/10; A61K 8/37; A61K 8/368; A61K 8/81; A61K 8/34

(52) U.S. Cl. CPC A61K 8/85; A61K 8/8152; A61K 8/362; A61K 8/345; A61K 8/375; A61K 8/368; A61Q 19/10; A61K 2800/28

(57) ABSTRACT

A method for producing a cosmetic cleaning agent includes: a) providing a preparation, which includes at least one acrylate homo-, co-, and/or crosspolymer in water, and heating and stirring the preparation at 30-40° C.; b) adding at least one antibacterial, antifungal, and/or antiseptic active substance, which is optionally dissolved, dispersed, or suspended in water and is selected from benzoic acid, salicylic acid, dehydroacetic acid, sorbic acid, cinnamic acid, and/or the physiologically acceptable salts of said acids, to the preparation from step a) and mixing the preparation from step a) with the at least one active substance; c) adding at least one surfactant, selected from anionic, amphoteric/zwitterionic, and/or nonionic surfactants, to the mixture from step b) and mixing the preparation from step b) with the at least one surfactant; d) optionally adding additional cosmetic active substances to the preparation from step c); and e) adding solid polylactic acid particles to the preparation from step c) or d).

### FIELD OF THE INVENTION

The present invention generally relates to cosmetics and relates to a method for producing a cleaning agent and a cosmetic cleaning agent.

### BACKGROUND OF THE INVENTION

Cosmetic cleaning agents have been long known and are improved regularly or adapted to changing consumer demands.

For example, consumers expect not only good cleansing and refreshing but also skin care properties from a modern cleaning agent. In particular, cleaning agents after their use on the skin should not leave behind a feeling of tightness and/or dryness. After cleaning, the skin should rather feel soft, smooth, and moisturized.

The cleaning of impure or oily skin or combination skin causes problems, insofar as many active substances used to combat impure or oily skin, have a very strong degreasing effect and can provoke even more rapid sebum production. The problem of the impure skin may be exacerbated even further thereby.

Likewise problematic is the cleaning and care of large-pored skin, which is regarded as unattractive and objectionable particularly on the face, in the neck and cleavage region, and on the upper back. Astringent active substances for refining skin pores are known from the prior art. These contract the skin on the surface and thus briefly create the impression of small-pored skin. At the same time, these agents nevertheless remove moisture from the skin, which is direly needed to maintain a taut skin with few wrinkles.

Accordingly, there is the need for cosmetic agents that are suitable for cleaning large-pored, impure skin or combination skin.

So-called scrubs are a variant of cosmetic cleaning agents especially preferred by many consumers for use on impure skin.

Cosmetic scrub products known from the prior art typically include abrasively acting particles such as polyethylene powder, walnut shell powder, or apricot or almond kernel powder. The dispersing and/or stabilizing of these solid active substances over a long time period and the preservation of the agents are a challenge, however.

Foaming body scrubs are described in WO 2011/149889 which include abrasive biodegradable lactic acid particles as the abrasive component. The lactic acid particles in this regard are incorporated into a base that includes an acrylate thickener and was neutralized beforehand to a pH of about 6 to 6.5. A pH in the neutral range within the scope of the application is important both for the thickening and for the preservation of the body scrubs, because they include formaldehyde-cleaving preservatives, especially effective at higher pH values.

In particular the cleaning and care of impure skin requires a treatment with mild compositions that are gentle on the skin and include no or the lowest possible amounts of potential skin-sensitizing active substances.

It is therefore desirable to provide a method for producing a mild cosmetic cleaning agent (particularly for the cleaning of and/or caring for impure skin), which allows the stabilization and/or dispersing of natural abrasive substances over a longer time period. It is also desirable to preserve the agents, without the stabilization and/or dispersing of the abrasive substances being lost as a result.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A method for producing a cosmetic cleaning agent includes the following steps: a) providing a preparation, which includes at least one acrylate homo-, co-, and/or crosspolymer in water, and heating and stirring the preparation at 30-40° C.; b) adding at least one antibacterial, antifungal, and/or antiseptic active substance, which is optionally dissolved, dispersed, or suspended in water and is selected from benzoic acid, salicylic acid, dehydroacetic acid, sorbic acid, cinnamic acid, and/or the physiologically acceptable salts of said acids, to the preparation from step a) and mixing the preparation from step a) with the at least one active substance; c) adding at least one surfactant, selected from anionic, amphoteric/zwitterionic, and/or nonionic surfactants, to the mixture from step b) and mixing the preparation from step b) with the at least one surfactant; d) optionally adding additional cosmetic active substances to the preparation from step c); and e) adding solid polylactic acid particles to the preparation from step c) or d).

A cosmetic cleaning agent includes, based on its total weight, 0.5 to 20% by weight of polylactic acid particles, which have absolute particle sizes in the range of 1 to 1000 μm; 0.01 to 10% by weight of at least one acrylate homo-, co-, and/or crosspolymers; and 0.01 to 1% by weight of at least one antibacterial, antimycotic, and/or antiseptic active substance, selected from benzoic acid, salicylic acid, dehydro acetic acid, sorbic acid, cinnamic acid, and/or the physiologically acceptable salts of said acids, whereby the cosmetic cleaning agent has a pH in the range of 4.5 to 5.8.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the invention is a method for producing a cosmetic cleaning agent, which has the following steps:
a) providing a preparation, which includes at least one acrylate homo-, co-, and/or crosspolymer in water, and heating and stirring the preparation at 30-40° C.;
b) adding at least one antibacterial, antifungal, and/or antiseptic active substance, which is optionally dissolved, dispersed, or suspended in water and is selected from benzoic acid, salicylic acid, dehydroacetic acid, sorbic acid, cinnamic acid, and/or the physiologically acceptable salts of said acids, to the preparation from step a) and mixing the preparation from step a) with the at least one active substance;
c) adding at least one surfactant, selected from anionic, amphoteric/zwitterionic, and/or nonionic surfactants, to the mixture from step b) and mixing the preparation from step b) with the at least one surfactant;
d) optionally adding additional cosmetic active substances to the preparation from step c); and
e) adding solid polylactic acid particles to the preparation from step c) or d).

It was found that the method of the invention assures the stabilization of the polylactic acid particles and the preserving of the cleaning agents, without additional preservatives (particularly no formaldehyde-cleaving preservatives) having to be incorporated, and without an alkalinization of the agents being absolutely necessary.

In a method preferred according to the invention, the cleaning agents resulting from the method therefore have a pH in the range of 4.5 to 5.8, preferably of 4.7 to 5.7, and especially preferably of 4.8 to 5.6.

An alkalinization step is not necessary in a preferred embodiment of the method according to the invention.

If it were to be necessary, nevertheless, to incorporate small amounts of an alkalinizing agent in a separate process step into the cleaning agent (in order to achieve a pH in the aforementioned range), then preferably a maximum of 0.45% by weight, more preferably a maximum of 0.35% by weight, particularly preferably a maximum of 0.25% by weight, and especially preferably a maximum of 0.15% by weight of an alkalinizing agent, preferably sodium hydroxide, would be necessary for this.

The stabilization and dispersing by the method according to the invention succeeds especially well, if at least one polymeric thickener, preferably a thickener with an acrylic acid (derivative) base, is used in step a). These are taken to mean acrylate homo-, co-, and/or crosspolymers, which can be preferably selected from crosslinked or non-crosslinked, hydrophobically modified polyacrylates and/or from crosslinked or non-crosslinked co- and/or crosspolymers of (meth)acrylic acid with at least one (meth)acrylic acid ester.

Preferably, these are anionic polymers that optionally can be hydrophobically modified.

Examples of anionic monomers which can comprise suitable acrylate homo-, co-, and/or copolymers are acrylic acid, methacrylic acid, crotonic acid, itaconic acid, maleic anhydride, and 2-acrylamido-2-methylpropanesulfonic acid. In this regard, the acid groups can be present entirely or partially as the sodium, potassium, ammonium, or the mono- or triethanolammonium salt. Preferred monomers are 2-acrylamido-2-methylpropanesulfonic acid and (meth)acrylic acid.

Preferred anionic homopolymers are non-crosslinked and crosslinked polyacrylic acids. In this regard, allyl ethers of pentaerythritol, of sucrose, and of propylene can be preferred crosslinking agents. Such compounds are, for example, available commercially under the trade name Carbopol®. Likewise preferred is the homopolymer of 2-acrylamido-2-methylpropanesulfonic acid, which is available commercially, for example, under the name Rheothik® 11-80.

Preferred furthermore are non-crosslinked and crosslinked, hydrophobically modified polyacrylic acids, which can be obtained from various suppliers as, for instance, 30% emulsions in water, for example, under the trade names Carbopol® Aqua SF1, Carbopol® Aqua SF2, or Rheomer® 33.

Preferred anionic acrylate copolymers are taken to mean copolymers of at least one anionic monomer and at least one nonionogenic monomer. In regard to the anionic monomers, reference is made to the substances cited above. Preferred nonionogenic monomers are acrylamide, methacrylamide, acrylic acid esters, (meth)acrylic acid esters, itaconic acid mono- and diesters, vinylpyrrolidone, vinyl ethers, and vinyl esters.

Preferred anionic copolymers are, for example, copolymers of acrylic acid, methacrylic acid, and/or C₁-C₆ alkyl esters thereof, as they are sold under the INCI declaration Acrylates Copolymer. Preferred commercial products are, for example, Aculyn® 33 from the company Rohm & Haas and/or Rheocare® TTA from the company Cognis. Preferred further are copolymers of acrylic acid, methacrylic acid, or C₁-C₆ alkyl esters thereof and the esters of an ethylenically unsaturated acid and an alkoxylated fatty alcohol. Suitable ethylenically unsaturated acids are in particular acrylic acid, methacrylic acid, and itaconic acid; suitable alkoxylated fatty alcohols are in particular Steareth-20 or Ceteth-20. Copolymers of this type are sold by Rohm & Haas under the trade name Aculyn® 22 and by the company National Starch under the trade names Structure® 2001 and Structure® 3001.

Preferred further are (meth)acrylic acid/C10-C30 alkyl acrylate copolymers, as they are commercially available, for example, under the trade name “Carbopol ETD 2020” (INCI name: Acrylates/C10-30 Alkyl Acrylate Crosspolymer).

The polymeric thickener(s) can be used in the method according to the invention preferably in amounts of 0.01 to 15% by weight, more preferably of 0.05 to 10% by weight, and especially of 0.1 to 10% by weight, whereby the quantitative data refer to the total weight of the cosmetic cleaning agent.

Suitable antibacterial, antimycotic, and/or antiseptic active substances within the scope of the method of the invention are to be taken to mean benzoic acid, salicylic acid, dehydroacetic acid, sorbic acid, cinnamic acid, and/or the physiologically acceptable salts of said acids.

In step b), the acids and/or acid salts can be added either directly to the preparation from step a) or dissolved, dispersed, or suspended in water.

Especially preferred are benzoic acid and/or salicylic acid and/or the alkali salts and/or alkaline earth salts of benzoic acid and/or salicylic acid. Sodium benzoate and/or sodium salicylate are especially preferred.

In a preferred embodiment of the method according to the invention, benzoic acid and/or salicylic acid and/or a physiologically acceptable salt of said acids are used in step b) as an antibacterial, antimycotic, and/or antiseptic active substance.

The antibacterial active substance(s) can be used in the method according to the invention preferably in amounts of 0.01 to 3% by weight, more preferably of 0.05 to 2% by weight, and especially preferably of 0.1 to 1% by weight.

It was found that aqueous emulsions or dispersions of the aforementioned crosslinked or non-crosslinked, hydrophobically modified acrylate homo-, co-, and/or crosspolymers in combination with the aforementioned acids or acid salts are especially suitable for use in the method according to the invention, because even at room temperature and pH values in the slightly acidic to neutral pH range they form gel networks by which the components insoluble in the cleaning agent (for example, abrasive substances) remain stably suspended.

The method according to the invention preferably results in a cosmetic cleaning agent that is aqueous or aqueous-alcoholic.

It is understood here that the agent includes preferably at least 50% by weight, more preferably at least 55% by weight, and especially preferably at least 60% by weight of water, whereby the quantitative data refer to the total weight of the cleaning agent.

Furthermore, the cleaning agent can include 0.01 to 30% by weight, preferably 0.05 to 35% by weight, and especially 0.1 to 30% by weight of at least one alcohol, which can be selected from ethanol, ethyl diglycol, 1-propanol, 2-propanol, isopropanol, 1,2-propylene glycol, glycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, sorbitol, benzyl alcohol, phenoxyethanol, or mixtures of said alcohols.

The water-soluble alcohols are preferred.

Apart from the abrasive properties, the method according to the invention should lead to cleaning agents that are mild for the skin and possess a good cleaning and skin care effect.

Conventional cosmetic cleaning agents because of their excellent cleaning and foaming ability include predominantly anionic surfactants, optionally in a mixture with low amounts of co-surfactants.

Many commercially available anionic surfactants soften the skin during the cleaning process and remove lipids from the outer skin layers. As a result, the skin can become dry, rough, and at times cracked, which is particularly undesirable in scrubs. On the other hand, anionic surfactants often cannot be completely replaced by milder, for example, nonionic surfactants, because the cleaning and foaming action of the agents is reduced thereby.

It was found that especially mild cleaning agents can be produced by the method according to the invention, if a maximum of 20% by weight of at least one mild anionic, amphoteric/zwitterionic, and/or zwitterionic surfactant is added in step c).

In another preferred embodiment, 0.5 to 20% by weight, more preferably 1 to 15% by weight, and especially preferably 2 to 12.5% by weight of at least one anionic, amphoteric/zwitterionic, and/or nonionic surfactant, preferably at least one amphoteric surfactant, are therefore added in step c). The quantitative data in this case refer to the total weight of the cosmetic cleaning agent.

Suitable anionic surfactants can be used in the method according to the invention preferably in amounts of 0.1 to 14.5% by weight, more preferably of 0.25 to 14% by weight, especially preferably of 0.5 to 12.5% by weight, and especially of 0.75 to 10% by weight, whereby the quantitative data refer to the total weight of the cleaning agent.

The suitable anionic surfactants include:
linear and branched fatty acids having 8 to 30 C atoms (soaps),
ether carboxylic acids of the formula R—O—(CH₂—CH₂O)ₓ—CH₂—COOH, in which R is a linear or branched, saturated or unsaturated alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and/or dialkyl esters having 8 to 24 C atoms in the alkyl group and sulfosuccinic acid monoalkylpolyoxyethyl esters having 8 to 24 C atoms in the alkyl group and 1 to 6 oxyethyl groups,
alpha-olefin sulfonates having 8 to 24 C atoms,
alkyl sulfate and/or alkyl polyglycol ether sulfate salts of the formula R—O(CH₂—CH₂O)ₓ—OSO₃⁻X⁺, in which R is a preferably linear or branched, saturated or unsaturated alkyl group having 8 to 30 C atoms, x=0 or 1 to 12, and X an alkali or ammonium ion,
sulfonates of unsaturated fatty acids having 8 to 24 C atoms and 1 to 6 double bonds,
esters of tartaric acid and citric acid with alcohols, representing addition products of approximately 2 to 15 molecules of ethylene oxide and/or propylene oxide to fatty alcohols having 8 to 22 C atoms,
alkyl and/or alkenyl ether phosphates of the formula,

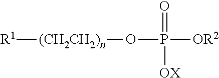

in which R¹ preferably stands for an aliphatic hydrocarbon group having 8 to 30 carbon atoms, R² for hydrogen, a group (CH₂CH₂O)ₙR¹, or X, n for numbers from 0 to 10, and X for hydrogen, an alkali metal or alkaline earth metal or NR³R⁴R⁵R⁶, where R³ to R⁶ independently of one another stand for a C₁ to C₄ hydrocarbon group.

Preferred anionic surfactants are ether carboxylic acids of the aforementioned formula, acyl sarcosides having 8 to 24 C atoms in the acyl group, sulfosuccinic acid mono- and/or -dialkyl esters having 8 to 24 C atoms in the alkyl group and sulfosuccinic acid monoalkylpolyoxyethyl esters having 8 to 24 C atoms in the alkyl group and 1 to 6 oxyethyl groups, alpha-olefin sulfonates having 8 to 24 C atoms, and/or alkyl sulfate salts and/or alkyl polyglycol ether sulfate salts of the aforementioned formula.

Especially preferred anionic surfactants are straight-chain or branched alkyl ether sulfates, which include an alkyl group having 8 to 18 and especially having 10 to 16 C atoms, and 1 to 6 and particularly 2 to 4 ethylene oxide units.

Furthermore, especially preferred anionic surfactants are straight-chain or branched alkyl sulfonates, which include an alkyl group having 8 to 18 and especially having 10 to 16 C atoms.

Preferred in particular are the sodium, magnesium, and/or triethanolamine salts of linear or branched lauryl, tridecyl, and/or myristyl sulfates, which have a degree of ethoxylation of 2 to 4.

Suitable amphoteric/zwitterionic surfactants can be used in the method according to the invention preferably in amounts of 0.1 to 14.5% by weight, more preferably of 0.25 to 14% by weight, especially preferably of 0.5 to 12.5% by weight, and especially of 0.75 to 10% by weight, whereby the quantitative data refer to the total weight of the cleaning agent.

Suitable amphoteric/zwitterionic surfactants can be selected from compounds of the following formulas (i) to (v), in which the group R in each case stands for a straight-chain or branched, saturated or mono- or polyunsaturated alkyl or alkenyl group having 8 to 24 carbon atoms,

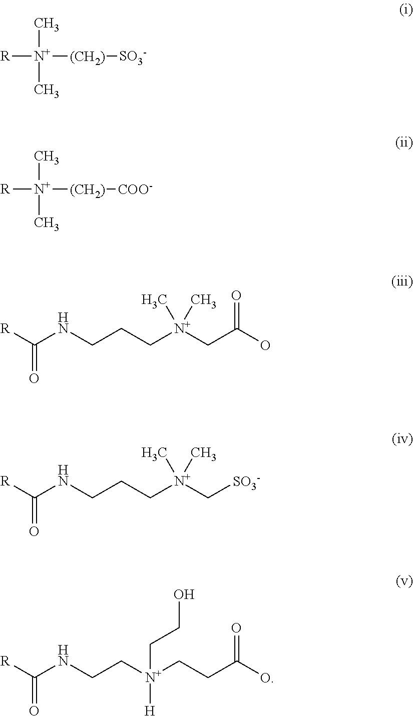

Especially suitable amphoteric/zwitterionic surfactants are alkyl amidoalkyl betaines and/or alkyl ampho(di)acetates of the aforementioned formulas (i) to (v).

Especially suitable amphoteric/zwitterionic surfactants include the surfactants known under the INCI names Cocamidopropyl Betaine and Disodium Cocoampho(di)acetate.

Suitable nonionic surfactants can be used in the method according to the invention preferably in amounts of 0.1 to 10% by weight, more preferably of 0.25 to 7.5% by weight, especially preferably of 0.5 to 6% by weight, and especially of 1 to 5% by weight, whereby the quantitative data refer to the total weight of the cleaning agent.

For the case that a nonionic surfactant is used in the method according to the invention, alkyl oligoglucosides, especially alkyl oligoglucosides based on hydrogenated C_{12/14} coconut alcohol or lauryl alcohol with a DP of 1-3, are preferred as they are commercially available, for example, under the INCI names “Coco-Glucoside” or “Lauryl Glucoside.”

Depending on the delivery form in which the cleaning agents resulting from the method according to the invention are offered, various aforementioned surfactants or surfactant combinations can be preferred.

For scrub compositions used during showering, for example, flowable, high-foaming compositions have proven especially suitable, which include
a mixture of anionic and amphoteric/zwitterionic surfactants in a weight ratio of 5:1 to 1:1, preferably of 4.5:1 to 2:1, or
a mixture of anionic, amphoteric/zwitterionic, and nonionic surfactants in a weight ratio of 5:1:0.5 to 1:1:0.5, preferably of 4.5:1:0.75 to 2:1:0.75.

Pasty compositions in particular are suitable as a scrub composition, which can be used specifically on facial skin, cleavage, the neck, or the back; these include
at least one amphoteric/zwitterionic surfactant, or
a mixture of at least one amphoteric/zwitterionic surfactant and at least one nonionic surfactant in a weight ratio of 5:1 to 1:5, preferably of 2:1 to 1:4.

To improve the skin care properties during and after use of the cleaning agents of the invention and/or to increase the abrasive and cleaning effect of the cleaning agents, it is advantageous, furthermore, in step d) of the method according to the invention to incorporate at least one active substance with a positive effect on skin moisture and/or at least one sebum-regulating and/or at least one skin-vitalizing active substance.

In another preferred embodiment, the method according to the invention therefore has the step d) in which added to the preparation from step c) are
0.001 to 20% by weight, more preferably 0.005 to 17.5% by weight, especially preferably 0.01 to 15% by weight, and exceedingly preferably 0.02 to 12.5% by weight of at least one active substance which has a positive effect on skin moisture and is selected from
   (i) freeze-dried yogurt powder,
   (ii) polyols,
   (iii) vitamins, and/or
   (iv) (optionally ethoxylated) mono-, di-, and/or triesters of glycerol with at least one C₈-C₂₄ fatty acid, and/or
0.001 to 5% by weight, more preferably 0.005 to 4% by weight, especially preferably 0.01 to 3% by weight, and exceedingly preferably 0.02 to 2% by weight of at least one sebum-regulating and/or at least one skin-vitalizing active substance, whereby the quantitative data refer to the total weight of the cosmetic cleaning agent.

“Freeze-dried yogurt powder” is understood to be a yogurt powder that can be obtained from natural yogurt (after complete fermentation) by freeze-drying.

An especially suitable yogurt powder, which can be used in the method according to the invention, preferably includes the following main components:
about 53.5% lactose,
about 25% proteins,
about 7.5% by weight of lactic acid,
about 5% mineral substances and trace elements,
about 1% vitamins, and
about 2% lipids.

Freeze-dried yogurt powder can be used in the method according to the invention preferably in an amount of 0.001 to 10% by weight, more preferably of 0.005 to 5% by weight, and especially of 0.01 to 3% by weight, whereby the quantitative data refer to the total weight of the cleaning agent.

A freeze-dried yogurt powder, known commercially under the name “Yogurtene®” is particularly suitable for use in the method according to the invention.

Suitable polyols are to be taken to mean preferably glycerol, 1,2-propylene glycol, 1,3-butylene glycol, hexanediol, sorbitol, water-soluble polysaccharides, polyethylene glycols, and/or hyaluronic acid. The polyols can be used both individually and as a mixture. Especially preferred are glycerol, 1,3-butylene glycol, hexanediol, and/or sorbitol, which may be used in the method according to the invention preferably in an amount of 0.1 to 20% by weight, more preferably of 0.5 to 15% by weight, and especially of 1 to 12.5% by weight.

Suitable vitamins are understood preferably as the following vitamins, provitamins, and vitamin precursors, and derivatives thereof:

Vitamin A: the group of substances designated as vitamin A include retinol (Vitamin A₁) and 3,4-didehydroretinol (vitamin A₂). Beta-carotene is the retinol provitamin. Suitable vitamin A components are, for example, vitamin A acid and esters thereof, vitamin A aldehyde, and vitamin A alcohol and esters thereof such as the palmitate and the acetate.
Vitamin B: The vitamin B group or the vitamin B complex includes, inter alia,
vitamin B₁ (thiamine)
vitamin B₂ (riboflavin)
Vitamin B₃. The compounds nicotinic acid and nicotinic acid amide (niacinamide) are often included under this term.
vitamin B₅ (pantothenic acid and panthenol). Panthenol is preferably used within the scope of this group. Usable panthenol derivatives are particularly the esters and ethers of panthenol and cationically derivatized panthenols. Individual representatives are, for example, panthenol triacetate, panthenol monoethyl ether, and the monoacetate thereof, as well as cationic panthenol derivatives.
vitamin B₆ (pyridoxine as well as pyridoxamine and pyridoxal).
   Vitamin C (ascorbic acid): the use in the form of the plamitic acid ester, glucosides, or phosphates can be preferred. The use in combination with tocopherols can likewise be preferred.
   Vitamin E (tocopherols, especially alpha-tocopherol).
   Vitamin F: The term “vitamin F” is conventionally understood to mean essential fatty acids, in particular linoleic acid, linolenic acid, and arachidonic acid.
   Vitamin H: Vitamin H is the name for the compound (3aS,4S,6aR)-2-oxohexahydrothienol[3,4-d]-imidazole-4-valeric acid, although the trivial name biotin has become accepted in the meantime.

Vitamins, provitamins, and vitamin precursors from groups A, B, E, and H can be used preferably in the method according to the invention.

Preferred in particular are nicotinic acid amide, biotin, pantolactone, and/or panthenol.

Vitamins, vitamin derivatives, and/or vitamin precursors can be used in the method according to the invention preferably in an amount of 0.001 to 2% by weight, more preferably of 0.005 to 1% by weight, and especially of 0.01 to 0.5% by weight, whereby the quantitative data refer to the total weight of the cleaning agent.

Suitable ethoxylated or non-ethoxylated mono-, di-, and/or tri-fatty acid esters of saturated and/or unsaturated linear and/or branched fatty acids with glycerol are typically used as an oil-replenishing agent for the skin and are accordingly suitable as a further active substance in the method according to the invention.

Especially preferred aforementioned glycerol esters can be selected from 2 to 30-fold, preferably from 3 to 20-fold, and especially from 5 to 10-fold ethoxylated mono-, di-, and/or tri-fatty acid esters of saturated and/or unsaturated linear and/or branched C₈-C₂₄ fatty acids, preferably of C₁₀-C₁₈ fatty acids and glycerol (for example, the components known under the INCI names: PEG-10 Olive Fatty Acid Glycerides, PEG-9 Coconut Fatty Acid Glycerides, Glycereth-5 Cocoate, PEG-7 Glyceryl Cocoate, PEG-6 Caprylic/Capric Glyceride). PEG-7 Glycerol Cocoate is very especially preferred.

The aforementioned (optionally ethoxylated) mono-, di-, and/or triesters of glycerol with at least one C₈-C₂₄ fatty acids can be used in the method of the invention preferably in amounts of 0.01 to 5% by weight, more preferably of 0.025 to 4% by weight, especially preferably of 0.05 to 3% by weight, and especially of 0.1 to 2% by weight, whereby the quantitative data refer to the total weight of the cleaning agents.

Suitable sebum-regulating active substances are taken to mean azelaic acid, azelaic acid derivatives, particularly potassium azeloyl diglycinate, which can be obtained, for example, as the commercial product Azeloglicina from Sinerga, sebacic acid, 10-hydroxydecanoic acid, 1,10-decanediol, mixtures of sebacic acid, 10-hydroxydecanoic acid, and 1,10-decanediol, as they can be obtained, for example, as the commercial product Acnacidol PG from Vincience, glycyrrhizin, which is also called glycyrrhizic acid or glycyrrhetinic acid glycoside, and 2-beta-glucuronido-alpha-glucuronide which represents glycyrrhetinic acid, and the salts thereof, tannic acid and the salts thereof, gallotannins, naringin, mixtures of glycyrrhizin (salts), tannic acid (salts), and/or gallotannins and naringin, as they can be obtained, for example, as commercial products BiSCos Glynarin PF (INCI: AQUA (WATER), ALCOHOL, PHENOXYETHANOL, AMMONIUM GLYGYRRHIZATE, TANNIC ACID, NARINGINE) from the company Biesterfeld, furthermore extracts from Spiraea ulmaria, as they are present, e.g., in the product Seboregul from the company Silab, furthermore water- and oil-soluble extracts from Hamamelis, burdock, and stinging nettle, cinnamon tree extract (e.g., Sepicontrol® A5 from the company Seppic), chrysanthemum extract (e.g., Laricyl® from Laboratoires Serobiologiques), yeast protein hydrolysates, as they can be obtained, e.g., in the commercial products of the Asebiol® series from Laboratoires Serobiologiques, particularly Asebiol® LS 2539 BT 2 (INCI: Aqua (water), Hydrolyzed Yeast Protein, Pyridoxine, Niacinamide, Glycerin, Panthenol, Allantoin, Biotin) and Asebiol® LS 2539 BT (Aqua (Water), Hydrolyzed Yeast Protein, Pyridoxine, Niacinamide, Glycerin, Panthenol, Propylene Glycol, Allantoin, Disodium Azelate, Biotin) and PEG-8 Isolauryl Thioether, as is included, e.g., in the commercial products “Antifettfaktor® COS-218/2-A” from Cosmetochem (INCI: Aqua (Water), Cetyl-PCA, PEG-8 Isolauryl Thioether, PCA, Cetyl Alcohol).

Methods especially preferred according to the invention are characterized in that at least one sebum-regulating active substance in a total amount of 0.001 to 5% by weight, preferably 0.01 to 2% by weight, especially preferably 0.05 to 1.5% by weight, and exceedingly preferably 0.1-0.5% by weight, in each case based on the overall cleaning agents, is added to them in step d).

Suitable skin-vitalizing active substances are understood to be preferably plant extracts that can be produced from all parts of a plant. These extracts are typically produced by extraction of the entire plant. It can also be preferred in individual cases, however, to produce the extracts solely from the flowers and/or leaves of the plant.

Preferred for use in the cleaning agents of the invention above all are the extracts from green tea, white tea, oak bark, stinging nettle, Hamamelis, hops, chamomile, burdock, horsetail, whitethorn, lime blossom, lychee, almond, aloe vera, spruce needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger root, Echinacea purpurea, Olea europaea, Foeniculum vulgaris, and Apium graveolens.

Water, alcohols, and mixtures thereof can be used as extracting agents to produce the cited plant extracts. Of the alcohols, low alcohols such as ethanol and isopropanol, but in particular polyhydric alcohols such as ethylene glycol and propylene glycol, are preferred, both as the sole extracting agent and in a mixture with water. Plant extracts based on water/propylene glycol in the ratio of 1:10 to 10:1 have proven to be particularly suitable.

The plant extracts can be used both in pure and diluted form. If they are used in diluted form, they conventionally include about 2 to 80% by weight of active substance and as the solvent, the extracting agent or mixture of extracting agents used to obtain them.

The plant extract(s) can be used in the method according to the invention preferably in amounts of 0.01 to 5% by weight, more preferably of 0.05 to 4% by weight, especially preferably of 0.1 to 3% by weight, and especially of 0.25 to 2% by weight, whereby the quantitative data refer to the total weight of the cleaning agent.

A biodegradable abrasive component is incorporated into the cosmetic cleaning agents (polylactic acid particles) in step e).

Polylactic acid, also called polylactide or PLA, is a name for biodegradable polymers (polyesters), which are obtainable primarily by the ionic polymerization of lactide, a ring-shaped joining of two lactic acid molecules.

A ring-opening polymerization occurs at temperatures between 140 and 180° C. and under the effect of catalytic tin compounds (e.g., tin oxide). Thus, plastics with a high molecular weight and strength are produced. Lactide itself can be produced by fermentation of molasses or by fermentation of glucose with the aid of various bacteria.

Moreover, high-molecular-weight and pure polylactides can be produced directly from lactic acid with the aid of so-called polycondensation. Nevertheless, the disposal of the solvent is problematic in industrial production.

Lactic acid (2-hydroxypropanoic acid) has an asymmetric C atom, so that polylactic acid as well has optically active centers in the L(+) and D(−) configuration. The ratio of L- to D-monomer units in this case determines the degree of crystallinity, the melting point, and the biodegradability of the polymers.

Polylactic acids suitable according to the invention are L-polylactic acid, D-polylactic acid, and L/D-polylactic acid, and mixtures thereof. L-polylactic acid is especially preferred because of its very good biodegradability. In a preferred embodiment of the present invention, the percentage by weight of the L-lactic acid monomer units in the polylactic acid is greater than 50% by weight, preferably greater than 80% by weight, and especially greater than 90% by weight.

The molar mass of the polylactic acid suitable according to the invention is preferably 1000 to 1,000,000, preferably 10,000 to 300,000, more preferably 50,000 to 250,000, and especially 100,000 to 180,000 daltons.

In another preferred embodiment of the present invention, polylactic acid is used in a form blended with fillers. The use of greater filler amounts is helpful in reducing the polymer into particles and increases the biodegradability and the inner specific surface via porosity and capillarity. In this case, water-soluble fillers are particularly preferred, for example, metal chlorides such as NaCl, KCl, etc., metal carbonates such as Na₂CO₃, NaHCO₃, etc., and metal sulfates such as MgSO₄.

Natural raw materials can also be used as fillers, for example, nut shells, wood or bamboo fibers, starch, xanthan gum, alginates, dextran, agar etc. These fillers are biodegradable and do not cause the good ecological properties of polylactic acid particles to worsen. The content of biodegradable fillers in the polylactic acid particles can be typically 10 to 70% by weight, whereby amounts of 20 to 60% by weight are preferred and those of 30 to 50% by weight are especially preferred.

Polylactic acid particles suitable according to the invention can be present both as spherical and as irregular particles, which have a specific circularity.

It is assumed that irregular shapes can intensify the abrasiveness of the polylactic acid particles; therefore, it can be advantageous for some embodiments of the present invention, if the polylactic acid particles preferably have a circularity between 0.1 and 0.6.

Polylactic acid particles with a lower circularity, in contrast, can be preferred, if a less abrasive, gentler abrading action of the cleaning agent according to the invention is to be achieved.

The shape of the polylactic acid particles employed according to the invention can be defined in various ways, whereby within the scope of this preferred embodiment of the present invention the geometric proportions of a particle and, more pragmatically, of a particle population are determined.

More recent, highly precise methods permit the precise determination of particle shapes from a large number of particles, typically of more than 10,000 particles, preferably of more than 100,000 particles. These methods enable a precise selection of the average particle shape of a particle population. The determination of particle shapes is preferably carried out with an “Occhio Nano 500 Particle Characterisation Instrument” with the software “Callistro version 25” (Occhio s.a. Liege, Belgium). This instrument enables the preparation, dispersing, imaging, and analysis of a particle population, whereby preferably the instrument parameters are set as follows: White Requested=180, vacuum time=5000 ms, sedimentation timer=5000 ms, automatic threshold, number of particle counted/analyses=8000 to 500,000, minimum number of replicates/sample=3, lens setting 1×/1.5×.

The polylactic acid particles, used according to the invention, preferably have sizes defined by their area-equivalent diameter (ISO 9276-6:2008(E) Section 7), also called “Equivalent Circle Diameter ECD” (ASTM F1877-05 Section 11.3.2). The mean ECD of a particle population is calculated as the mean ECD of each individual particle of a particle population of at least 10,000 particles, preferably of more than 50,000 particles, especially of more than 100,000 particles, after particles with an area-equivalent diameter (ECD) below 10 μm were excluded from the measurement.

In a preferred embodiment of the present invention, the polylactic acid particles have mean ECD values of 10 to 1000 μm, preferably of 50 to 500 μm, more preferably of 100 to 350 μm, and especially of 150 to 250 μm.

Independent of the average particle size, methods according to the invention are preferred in which the polylactic acid particles used in step e) have absolute particle sizes of 1 to 1000 μm, more preferably of 1 to 850 μm, particularly preferably of 1 to 750 μm, exceedingly preferably of 1 to 500 μm, and especially preferably of 1 to 300 μm.

Within the scope of the present invention, shape descriptors are used which are calculations of geometric descriptors or shape factors. Shape factors are ratios between two different geometric properties, which for their part are a measurement of the proportions of the image of a whole particle or the measurement of the proportions of an ideal geometric body, enveloping the particle.

These results are descriptors similar to size ratios (aspect ratios). In a preferred embodiment of the present invention, mesoshape descriptors are used for particle characterization. These mesoshape descriptors indicate the extent to which a particle deviates from an ideal geometric shape, particularly from a sphere.

In a first preferred embodiment of the present invention, the polylactic acid particles can deviate from the typical spherical shape or sphere-like shapes such as, for example, granular particles (see above).

In this case, the particles preferably have sharp corners and edges and preferably possess concave curvatures. Sharp corners of non-spherical particles in this regard are defined by a radius less than 20 μm, preferably less than 8 μm, and especially less than 5 μm, whereby the radius is defined as the radius of an imaginary circle that follows the contour of the corner.

Circularity is a quantitative, 2-dimensional image analysis and can be determined according to ISO 9276-6:2008(E) Section 8.2. Circularity is a preferred mesoshape descriptor and can be determined, for example, with the above-described “Occhio Nano 500 Particle Characterisation Instrument” with the software “Callistro version 25” (Occhio s.a. Liege, Belgium) or with the “Malvern Morphologi G3.” Circularity is occasionally described in the literature as the difference between a particle and the perfect spherical shape. The values for circularity vary between 0 and 1, whereby 1 describes the perfect sphere or (in the two-dimensional image) the perfect circle:

C=[(4πA(/p²]^{1/2}

where A is the projection area (the two-dimensional descriptor) and p the length of the perimeter of the particle.

Within said preferred embodiment, polylactic acid particles with a mean circularity C of 0.1 to 0.6, preferably of 0.15 to 0.4, and especially of 0.2 to 0.35 have proven especially suitable within the scope of the present invention. In this case, the mean values are obtained by quotient formation from volume-based measurements and number-based measurements.

Solidity is a quantitative, 2-dimensional image analysis and can be determined according to ISO 9276-6:2008(E) Section 8.2. Solidity is likewise a preferred mesoshape descriptor and can be determined, for example, with the above-described “Occhio Nano 500 Particle Characterisation Instrument” with the software “Callistro version 25” (Occhio s.a. Liege, Belgium) or with the “Malvern Morphologi G3.” Solidity is a mesoshape descriptor, which describes the concavity of a particle or a particle population. Solidity values vary between 0 to 1, whereby 1 describes a non-concave particle:

Solidity=A/Ac

where A is the (image) area of the particle and Ac is the area of the convex shell enveloping the particle.

Within the first preferred embodiment, polylactic acid particles having a mean solidity of 0.4 to 0.9, preferably of 0.5 to 0.8, and especially of 0.55 to 0.65 have proven to be especially suitable within the scope of the invention. In this case, the mean values are obtained by quotient formation from volume-based measurements and number-based measurements.

Especially preferred polylactic acid particles of the first preferred embodiment preferably have a mean circularity C of 0.1 to 0.6, preferably of 0.15 to 0.4, and especially of 0.2 to 0.35 and a mean solidity of 0.4 to 0.9, preferably of 0.5 to 0.8, and especially of 0.55 to 0.65.

“Mean” circularity and solidity are averages from the measurement of a large number ofparticles, typically of more than 10,000 particles, preferably of more than 50,000 particles, and especially of more than 100,000 particles, whereby particles with an area-equivalent diameter (ECD) of less than 10 μm were excluded from the measurement.

After its preparation, the polylactic acid polymer can be converted to the desired particle size and shape, for example, by a grinding process, depending on the shape required for the particular purpose.

An especially preferred method for preparing polylactic acid particles with the desired circularity and solidity consists of preparing a foam from polylactic acid and subsequent grinding.

It is assumed that a specific hardness can enhance the abrasive effect of the polylactic acid particles; therefore, it can be advantageous for some embodiments of the present invention, furthermore, if the polylactic acid particles have hardnesses of 3 to 50 kg/mm², preferably of 4 to 25 kg/mm², and especially of 5 to 15 kg/mm² on the HV Vickers hardness scale.

The hardness of the particles in this case can be varied via the ratio of the D- to L-monomers and via the molar mass.

Polylactic acid particles, which can be used preferably in the method of the invention, are commercially available (for example, from the company Micro Powders, Inc., under the trade names Ecosrub®). Especially preferred are the commercial products Ecosrub® 20PC, Ecosrub® 50PC, Ecosrub® 100PC, Ecoblue® 5025, and Ecogreen® 5025. Preferred in particular are Ecosrub® 20PC and Ecosrub® 50PC.

The polylactic acid particles are used in step e) preferably in an amount of 0.5 to 20% by weight, more preferably of 1 to 15% by weight, and especially preferably 1 to 8% by weight, whereby the quantitative data refer to the total weight of the cleaning agent.

To achieve a wide range of textures, it is advantageous, furthermore, if at least one opacifier and/or at least one pearlescent agent is incorporated, furthermore, in the method according to the invention.

The opacifier/pearlescent agent in this case can be incorporated as a dispersion or suspension in water, or in pure form in one of the steps a) to d). Preferred use amounts of the opacifiers and/or pearlescent agents are 0.01 to 5% by weight, more preferably 0.05 to 4% by weight, especially preferably 0.1 to 3% by weight, and especially 0.2 to 2% by weight, whereby the quantitative data refer to the total weight of the cleaning agent.

Suitable pearlescent agents and opacifiers are taken to mean, for example,
mono- and/or diesters of ethylene glycol, 1,2-propanediol, and/or glycerol with C₈-C₂₄ fatty acids,
inorganic pigments,
esters of polyethylene glycols with C₈-C₂₄ fatty acids, and/or
styrene/acrylate copolymers.

Especially suitable are the opacifiers and/or pearlescent agents known under the INCI names:

titanium dioxide, synthetic and/or natural mica pigments coated with metal oxide(s), glycol distearate, such as, for example, the commercial product Cutina® AGS from the company Cognis, glycol monostearate, such as, for example, the commercial product Cutina® EGMS from the company Cognis, PEG-3 distearate, such as, for example, the commercial product Genapol® TS from the company Clariant, PEG-2 distearate, such as, for example, the commercial product Kessco® DEGMS from the company AkzoNobel, propylene glycol stearate, such as, for example, the commercial product Tegin® P from the company Goldschmidt, and/or styrene/acrylate copolymers, such as, for example, the commercial products Joncryl® 67 from the company Johnson Polymers, Suprawal® WS from the company BASF, and/or Acusol® OP 301 from the company Rohm & Haas.

To increase the mildness and care properties, it is advantageous, furthermore, if preferably 0.01 to 5% by weight, more preferably 0.05 to 4% by weight, especially preferably 0.075 to 3% by weight, and especially 0.1 to 2% by weight of at least one cationic polymer are added to the cleaning agent in step d). The quantitative data in this case refer to the total weight of the cleaning agent.

Suitable cationic polymers are, for example:
quaternized cellulose derivatives, as they are commercially available under the names Celquat® and Polymer JR®,
hydrophobically modified cellulose derivatives, for example, the cationic polymers sold under the trade name SoftCat®,
cationic alkyl polyglycosides,
cationized honey, for example, the commercial product Honeyquat® 50,
cationic guar derivatives, such as in particular the products sold under the trade names Cosmedia® Guar, N-Hance®, and Jaguar®,
polymeric dimethyldiallylammonium salts and the copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as, for example, vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as they are sold under the names Luviquat® FC 370, FC 550, FC 905, and HM 552,
quaternized polyvinyl alcohol,
   as well as polymers known under the names
Polyquaternium 2, Polyquaternium 17, Polyquaternium 18, Polyquaternium-24, Polyquaternium 27, Polyquaternium-32, Polyquaternium-37, Polyquaternium 74, and Polyquaternium 89.

Preferred cationic polymers are quaternized cellulose polymers, cationic guar derivatives, and/or cationic polymers with an acrylic acid (derivative) base, which are selected especially preferably from the polymers known under the INCI names: Guar Hydroxypropyltrimonium Chloride, Polyquaternium-6, Polyquaternium-7, Polyquaternium-10, Polyquaternium-37, and/or Polyquaternium-67.

A cationic polymer known under the INCI name Polyquaternium-7 is especially preferred for use in the cleaning agents of the invention.

Methods preferred according to the invention lead to cosmetic cleaning agents, which can have viscosities in the range of 3000 to 400,000 mPas, depending on whether these involve a flowable shower scrub or a pasty scrub, for example, for cleaning facial skin.

In a preferred embodiment, the method according to the invention leads to a shower scrub, which has a viscosity especially preferably in the range of 4000 to 30,000 mPas and especially of 5000 to 20,000 mPas (in each case measured using a Haake rotational viscometer VT550, 20° C., measuring device MV, spindle MV II, 8 rpm).

In another preferred embodiment, the method according to the invention leads to a pasty composition, which has a viscosity especially preferably in the range of 100,000 to 400,000 mPas and especially of 200,000 to 400,000 mPas (in each case measured with a Brookfield rotational viscometer RVTDV II, 20° C., rotation speed 4 min-1, spindle No.: TC, Helipath).

Other active substances, auxiliary substances, and additives, which can be used in the method according to the invention, are, for example:
physiologically acceptable oil, wax, and/or fat components,
UV filters,
thickeners, such as gelatin or plant gum, for example, agar-agar, guar gum, alginates, xanthan gum, gum arabic, karaya gum, locust bean gum, flaxseed gums, dextrans, cellulose derivatives, e.g., methyl cellulose, hydroxyalkyl cellulose, and carboxymethyl cellulose, starch fractions and derivatives such as amylose, amylopectin, and dextrins, clays and phyllosilicates such as, e.g., bentonite, or fully synthetic hydrocolloids such as, e.g., polyvinyl alcohol, and Ca, Mg, or Zn soaps,
structurants such as maleic acid and lactic acid,
dyes for coloring the agent,
substances for adjusting the pH, for example, α- and β-hydroxycarboxylic acids such as citric acid, lactic acid, malic acid, and glycolic acid,
active substances such as bisabolol,
complexing agents such as EDTA, NTA, beta-alanine diacetic acid, and phosphonic acids,
antioxidants,
other viscosity regulators such as electrolyte salts (NaCl).

The method according to the invention has the advantage that it is suitable for producing mild abrasive cleaning agents. The stabilization of polylactic acid and the preserving of the agent are achieved by a combination of special acrylate thickeners with special acidic preservatives (salts), as a result of which an additional alkalinization step is no longer absolutely necessary. The cleaning agents obtainable by the method according to the invention are particularly suitable for the gentle and thorough cleansing/care of impure skin.

A second subject of the invention is cosmetic cleaning agents, including, based on their total weight,
0.5 to 20% by weight of polylactic acid particles, which have absolute particle sizes in the range of 1 to 1000 μm,
0.01 to 10% by weight of at least one acrylate homo-, co-, and/or crosspolymers, and
0.01 to 1% by weight of at least one antibacterial, antimycotic, and/or antiseptic active substance, selected from benzoic acid, salicylic acid, dehydro acetic acid, sorbic acid, cinnamic acid, and/or the physiologically acceptable salts of said acids,
   whereby the cosmetic cleaning agent has a pH in the range of 4.5 to 5.8.

A third subject of the invention is the use of the above-described cosmetic cleaning agent for cleaning the skin and/or for improving the complexion, particularly for pore refinement in the facial, back, and cleavage region and/or for matting the skin.

The statements made about the method according to the invention apply mutatis mutandis to other preferred embodiments of the cleaning agent according to the invention and the use according to the invention.

### EXAMPLES

The following skin-refining scrubs were prepared:

| | 1 | 2 |
|---|---|---|
| Carbopol ®¹ ETD 2020 | 0.8 | 1 |
| Sorbitol 70% | 15 | 15 |
| Disodium cocoamphodiacetate (40% active substance) | 2.4 | 2.5 |
| Plantacare ®² 1200 UP | 5 | 5 |
| PEG-7 Glyceryl Cocoate | 1 | 1.5 |
| White tea extract | 0.5 | |
| Ginseng extract | 0.45 | 0.5 |
| Seboregul ®³ | 0.1 | 0.1 |
| Sodium benzoate | 0.4 | 0.4 |
| Titanium dioxide | 0.4 | |
| Ecoscrub ®⁴ 50PC | 3 | |
| Ecoscrub ®⁴ 20PC | | 2 |
| Oeresundsand ®⁵ 18 | 7 | 6 |
| Citric acid, NaOH | q.s. | q.s. |
| Water | To 100 | To 100 |
| pH | 4.8-5.4 | 4.8-5.4 |

The following commercial products were used in the facial skin scrubs:
¹INCI name: Acrylates/C10-30 Alkyl Acrylates Crosspolymer; Lubrizol,
²INCI name: Lauryl Glucoside, Aqua; BASF,
³INCI name: Butylene Glycol, Aqua, Spiraea Ulmaria Extract; Silab
⁴INCI name: Polylactic Acid; Micro Powders, Inc.
⁵INCI name: INCI name: Quartz; Askania AB

The production method according to the invention is described with use of Example 1:

1) Carbopol® ETD 2020 in water was charged into a pot at 30-40° C. and stirred until clumps were no longer present.
2) Next, sodium benzoate in pure form was added to the pot and stirred until the sodium benzoate was completely dissolved.
3) This was followed by the addition of disodium cocoamphodiacetate and Plantacare 1200.
4) Next, all components in the table were stirred in, except for the polylactic acid particles, the Oeresundsand, and the titanium dioxide particles, until a homogeneous mixture formed. The mixture was deaerated for about 20 minutes.
5) Titanium dioxide was presuspended in water, added to the mixture from step 4), and stirred to homogeneity. Next, the mixture was deaerated again for about 15 minutes.
6) In the last step, the polylactic acid particles and the Oeresundsand were added with very gentle stirring.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for producing a cosmetic cleaning agent, which includes:
a. providing a preparation, which includes at least one acrylate homo-, co-, and/or crosspolymer in water, and heating and stirring the preparation at 30-40° C.;
b) adding at least one antibacterial, antifungal, and/or antiseptic active substance, which is selected from the group consisting of benzoic acid, salicylic acid, dehydroacetic acid, sorbic acid, cinnamic acid, and the physiologically acceptable salts of such acids, to the preparation from step a) and mixing the preparation from step a) with the at least one active substance;
c) adding at least one surfactant, selected from anionic, amphoteric/zwitterionic, and nonionic surfactants, to the mixture from step b) and mixing the preparation from step b) with the at least one surfactant;
d) optionally adding additional cosmetic active substances to the preparation from step c); and
e) adding solid polylactic acid particles to the preparation from step c) or d).

2. The method according to claim 1, wherein the cosmetic cleaning agent has a pH in the range of 4.5 to 5.8.

3. The method according to claim 1, wherein the acrylate homo-, co-, and/or crosspolymer is selected from the group consisting of crosslinked or non-crosslinked, hydrophobically modified polyacrylates and crosslinked or non-crosslinked co- and/or crosspolymers of (meth)acrylic acid with at least one (meth)acrylic acid ester.

4. The method according to claim 3, wherein the acrylate homo-, co-, and/or crosspolymer is an anionic polymer that can be hydrophobically modified.

5. The method according to claim 1, wherein the polymeric thickener is used in an amount of 0.01 to 15% by weight, referring to the total weight of the cosmetic cleaning agent.

6. The method according to claim 1, wherein 0.5 to 20% by weight of at least one anionic, amphoteric/zwitterionic, and/or nonionic surfactant is added in step c), referring to the total weight of the cosmetic cleaning agent.

7. The method according to claim 1, wherein added to the preparation from step c) in step d) are
0.001 to 20% by weight of at least one active substance which has a positive effect on skin moisture and selected from
(i) freeze-dried yogurt powder,
(ii) polyols,
(iii) vitamins, and
(iv) optionally ethoxylated mono-, di-, and/or triesters of glycerol with at least one C₈-C₂₄ fatty acid, and/or
0.001 to 5% by weight of at least one sebum-regulating and/or at least one skin-vitalizing active substance,
referring to the total weight of the cosmetic cleaning agent.

8. The method according to claim 1, wherein added to the preparation from step c) or d) in step e) are polylactic acid particles, which have absolute particle sizes in the range of 1 to 1000 μm.

9. The method according to claim 8, wherein the polylactic acid particles are used in an amount of 0.5 to 20% by weight referring to the total weight of the cosmetic cleaning agent.

10. The method according to claim 1, wherein the antibacterial, antimycotic, and/or antiseptic active substance in step b) is one or more selected from the group consisting of benzoic acid, salicylic acid, and physiologically acceptable salts of such acids.

11. The method according to claim 1, wherein the antibacterial, antimycotic, and/or antiseptic active substance is used in step b) in amounts of 0.01 to 3% by weight referring to the total weight of the cosmetic cleaning agent.

12. The method according to claim 1, wherein the cosmetic cleaning agent is a shower scrub having a viscosity in the range of 4000 to 30,000 mPas measured using a Haake rotation viscometer VT550, 20° C., measuring device MV, spindle MV II, 8 rpm.

13. The method according to claim 1, wherein the cosmetic cleaning agent is a pasty composition having a viscosity in the range of 100,000 to 400,000 mPas measured using a Brookfield rotational viscometer RVTDV II, 20° C., rotation speed 4 min-1, spindle No. TC, Helipath.

14. A cosmetic cleaning agent, including, based on its total weight,
0.5 to 20% by weight of polylactic acid particles, which have absolute particle sizes in the range of 1 to 1000 μm,
0.01 to 10% by weight of at least one acrylate homo-, co-, and/or crosspolymers, and
0.01 to 1% by weight of at least one antibacterial, antimycotic, and/or antiseptic active substance, selected from the group consisting of benzoic acid, salicylic acid, dehydro acetic acid, sorbic acid, cinnamic acid, and the physiologically acceptable salts of such acids,
wherein the cosmetic cleaning agent has a pH in the range of 4.5 to 5.8.
